(19) United States
(12) Patent Application Publication
(10) Pub. No.: US 2026/0169003 A1
(43) Pub. Date: Jun. 18, 2026

(54) PROTEIN DETECTION METHOD BASED ON COMBINED NANOPORE STRUCTURE

(71) Applicant: Guangdong University of Technology, Guangzhou (CN)

(72) Inventors: Zhishan YUAN, Guangzhou (CN); Hongwen WU, Guangzhou (CN); Derong XU, Guangzhou (CN)

(73) Assignee: Guangdong University of Technology, Guangzhou, GD (CN)

(21) Appl. No.: 19/533,770

(22) Filed: Feb. 9, 2026

(51) Int. Cl. G01N 33/68; G01N 33/487

(52) U.S. Cl. CPC G01N 33/6842; G01N 33/48721; G01N 33/6803; G01N 33/6848

(57) ABSTRACT

The present disclosure relates to the field of protein detection technology, and discloses a protein detection method based on a combined nanopore structure, comprising: a surface of a sandwich film chip is processed to form a nano-through hole, and a “hole-cavity-hole” structure is prepared. The “hole-cavity-hole” structure is driven by the electric field of the functional protein to detect the protein. The present disclosure adopts the above-mentioned protein detection method based on a combined nanopore structure, which can effectively overcome the problem of protein detection reading length caused by the limitation of nanopore space. Meanwhile, in contrast to methods that embed functional protein complexes within a phospholipid bilayer, integrating functional proteins directly into solid nanopores combines the advantages of both biological and solid systems. This approach not only relaxes the stringent environmental demands placed on the sensor but also enhances its resolution and stability.

### TECHNICAL FIELD

The present disclosure relates to the field of protein detection technology, in particular to a protein detection method based on a combined nanopore structure.

### BACKGROUND

Modern proteomics relies heavily on tandem mass spectrometry (MS) technology, renowned for its high precision and ability to identify and quantify proteins within complex mixtures. However, most mass spectrometers are bulky, involve substantial initial investment and maintenance costs, and require specialized operational expertise. With the growing demand for high-throughput proteomic research and personalized medicine, there is an urgent need to develop scalable, low-cost protein analysis techniques.

In contrast to mass spectrometry instrumentation, nanopore-based detection offers a low-cost, high-throughput platform capable of operating under native conditions. In nanopore analysis, when an analyte traverses a single nanopore, an applied potential induces a transient blockage of the ionic current through the pore. Crucially, the extent of current blockade is largely proportional to the volume excluded by the analyte, thereby enabling size-based discrimination among chemically similar (bio)polymers such as PEG chains, DNA, proteins, and peptides. Moreover, nanopores allow accurate, label-free, rapid, and high-precision detection of various molecules, including proteins and DNA, at the single-molecule level.

Currently, biological nanopore technology has been demonstrated to achieve highly sensitive detection and resolution of all 20 amino acids. Recently, Martin-Baniandres et al.^{[1]} utilized an engineered charge-selective nanopore to accomplish non-enzymatic capture, unfolding, and translocation of a single polypeptide chain via electroosmotic flow, thereby enabling the detection of post-translational modifications within the polypeptide. The length of these polypeptides can exceed 1200 amino acid residues, suggesting the potential of nanopores for long-read protein sequencing. Nova et al.^{[2]} employed Hel308 helicase to control peptide translocation through the nanopore sensing region, achieving single-molecule detection of phosphorylation modifications and distinguishing peptides containing one or two adjacent phosphorylation sites with 95% accuracy. Nevertheless, due to the spatial constraints of the nanopore, the detectable length in this approach is limited to 15-20 amino acids. Motone et al.^{[3]} used ClpX unfoldase with CsgG nanopores to thread entire protein molecules, enabling long-range single-molecule reading and complete protein chain sequencing. However, this method involves pretreatment of the target peptide and depends on ClpX binding efficiency, which somewhat restricts its broader applicability. Thus, protein (peptide) sequencing still faces challenges, including short read lengths, limited stability, and insufficient accuracy. Consequently, achieving stable, long-read, and highly accurate protein sequencing remains of great significance for life sciences research.

Accordingly, this application proposes a combined nanopore-based protein detection device and sequencing method to address the above technical limitations.
^{[1]}Martin-Baniandres, P., Lan, W H., Board, S. et al. Enzyme-less nanopore detection of post-translational modifications within long polypeptides. Nat. Nanotechnol. 18, 1335-1340 (2023).
^{[2]}Nova, I. C., Ritmejeris, J., Brinkerhoff, H. et al. Detection of phosphorylation post-translational modifications along single peptides with nanopores. Nat Biotechnol42, 710-714 (2024).
^{[3]}Motone, K., Kontogiorgos-Heintz, D., Wee, J. et al. Multi-pass, single-molecule nanopore reading of long protein strands. Nature 633, 662-669 (2024).

### SUMMARY

The purpose of this present disclosure is to provide a protein detection method based on a combined nanopore structure, which can effectively overcome the problem of protein detection reading length caused by the limitation of nanopore space. Meanwhile, in contrast to methods that embed functional protein complexes within a phospholipid bilayer, integrating functional proteins directly into solid nanopores combines the advantages of both biological and solid systems. This approach not only relaxes the stringent environmental demands placed on the sensor but also enhances its resolution and stability.

In order to achieve the above purpose, the present disclosure provides a protein detection method based on a combined nanopore structure, including the following steps:
1) Construction of a protein detection device with a combined nanopore structure:
Step 1, processing the surface of the sandwich film chip and milling to form nano-through holes;
Step 2, preparation of the “hole-cavity-hole” structure: tilting the sandwich film chip with nanopores prepared in Step 1 in BOE solution, and using the buffer oxide etchant to etch the intermediate layer to obtain a sandwich film chip with a “hole-cavity-hole” structure;
Step 3, embedding the functional protein in the sandwich film chip with the “hole-cavity-hole” structure by an electric field: washing the sandwich film chip obtained in Step 2 with piranha solution and loading into the liquid pool chamber, filling the both sides of the liquid pool chamber with the electrolyte containing two functional proteins, and applying a bias voltage, under the action of electric field force, embedding the two functional proteins in the solid nanopores on both sides of the sandwich film chip, and obtaining the protein detection device based on the combined nanopore structure.
2) Protein detection:

Functional proteins that control the translocation speed of biomolecules drive peptide-modified DNA molecules to read and detect peptide sequences in peptide-modified DNA molecules through proteins with high-resolution reading ability, when the peptide sequence enters the protein with high-resolution reading ability, the reading of the peptide sequence shows a change in the current signal.

In some embodiments, when etching in step 2, the volume of the middle layer cavity is adjusted by controlling the etching time. The etching time is 0.5 s-30 min, and the volume of the middle layer cavity is 100 nm³-1×10⁵ nm³.

In some embodiments, the two functional proteins in Step 3 are functional proteins that control the translocation speed of biomolecules and proteins with high-resolution reading ability; preferably, the functional proteins that control the rate of translocation of biomolecules include phi29DNAP, hel308, ClpX helicase, etc. Proteins with high-resolution reading ability include MspA, α-HL, SP1, aerolysin, Phi29, CsgG, SPP1, FraC, etc.

The detection principle of the present disclosure is as follows:

The functional protein controlling the translocation speed of biomolecules drives the peptide-modified DNA molecule through the protein with high-resolution reading capability, enabling the reading and detection of the peptide chain sequence carried on the DNA molecule. Between the two functional proteins lies a tunable cavity layer whose thickness can be adjusted during the manufacturing process, thereby allowing the detection of proteins of varying lengths. This design effectively overcomes the limitation on protein detection read length imposed by the spatial constraints of the nanopore. When the peptide chain sequence enters the high-resolution reading protein, the reading process manifests as a change in the recorded current signal.

The advantages and beneficial effects of the protein detection method based on the combined nanopore structure described in the present disclosure are as follows:

In this present disclosure, a cavity exists between the two functional proteins, and its thickness can be precisely adjusted via the fabrication process. The resulting combined nanopore structure effectively addresses the restriction in protein detection read length caused by the limited spatial dimensions of conventional nanopores. Moreover, compared to methods that embed functional protein complexes into a phospholipid bilayer, the present approach incorporates functional proteins directly into a solid nanopore. This integration combines the advantages of biological and solid nanopores, not only relaxing the stringent environmental requirements for sensor operation but also enhancing both resolution and stability.

The technical solution of the present disclosure is further described in detail below with reference to the accompanying drawings and implementation examples.

### BRIEF DESCRIPTION OF THE DRAWINGS

FIG. 1 is a schematic diagram of the sandwich film chip structure in the embodiment of the present disclosure;

FIG. 2 is a schematic diagram of protein molecule detection in the embodiment of the present disclosure;

FIG. 3 is a current change diagram of the protein sequencing stage in the embodiment of the present disclosure, wherein (1) is the current diagram of the sandwich film chip, (2) is the current diagram of the sandwich film chip embedded with a protein with high-resolution reading ability, (3) and (4) are the current diagrams of the sandwich film chip embedded with a protein with high-resolution reading ability and embedding with a protein with control of the translocation speed of biomolecules, (5) and (6) are the current diagrams of the protein sequencing.

### Marks in the Diagrams

1, sandwich film chip; 2, phi29DNAP; 3, MspA.

### DETAILED DESCRIPTION OF THE EMBODIMENTS

The following is a further explanation of the technical scheme of the present disclosure through drawings and implementation examples.

Unless otherwise defined, the technical terms or scientific terms used in the present disclosure should be understood by people with general skills in the field to which the present disclosure belongs.

Unless otherwise defined, the instruments and reagents used in the present disclosure are conventionally commercially available.

The combined nanopore protein sequencing method includes the following steps:
Step 1, the surface of the sandwich film chip is processed and milled to form nano-through holes; the processing method is not limited, and the existing processing method is selected according to the needs until the nanopore is drilled.
Step 2, preparation of “hole-cavity-hole” structure: the sandwich film chip with nanopores prepared in Step 1 is tilted in BOE solution, and the buffer oxide etchant is used to etch the intermediate layer to obtain a sandwich film chip with “hole-cavity-hole” structure; the optimal etching time is 3-5 min, and the volume of the interlayer cavity is 100 nm³-1×10⁵ nm³.
Step 3, the functional protein electric field drives the sandwich film chip embedded in the “hole-cavity-hole” structure: the sandwich film chip obtained in Step 2 is washed with piranha solution and loaded into the liquid pool chamber, the electrolyte containing two functional proteins is filled on both sides of the liquid pool chamber, and the bias voltage is applied, under the action of electric field force, the two functional proteins are embedded in the solid nanopores on both sides of the sandwich film chip; the two functional proteins are functional proteins that control the translocation speed of biomolecules and proteins with high-resolution reading ability. Functional proteins that control the speed of biomolecular translocation include DNA synthase, DNA helicase, DNA topoisomerase, such as phi29DNAP, hel308, ClpX helicase, etc. Proteins with high-resolution reading ability include MspA, α-HL, SP1, aerolysin, Phi29, CsgG, SPP1, FraC, etc.

A protein with high-resolution reading ability for biomolecules is embedded into the first solid nanopore, and then another protein with the function of controlling the translocation speed of peptide-modified DNA molecules is embedded into the second solid nanopore driven by the electric field force (the protein binds to the peptide-modified DNA molecules before embedding the solid nanopore). A combined nanopore protein detection device is formed, and then combined nanopore protein sequencing is performed.
Step 4, sequence read length detection: Proteins with biomolecular motor function drive peptide-modified DNA molecules to detect the target peptide sequence through proteins with high-resolution read ability.

The following sandwich film chip material is Si₃N₄/SiO₂/Si₃N₄, select phi29DNAP as a functional protein to control the translocation speed of DNA molecules, and select MspA to read biomolecules. The protein is used as an example to explain the application in detail.

### Embodiment

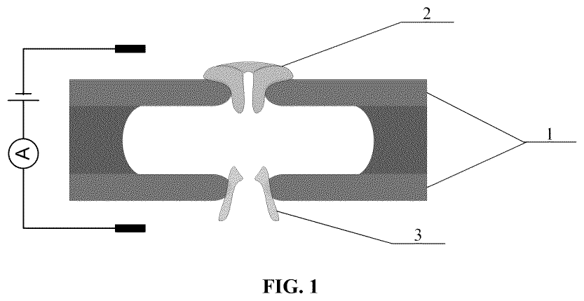

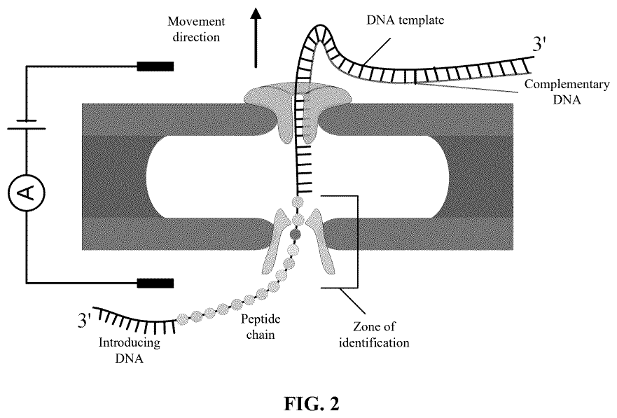

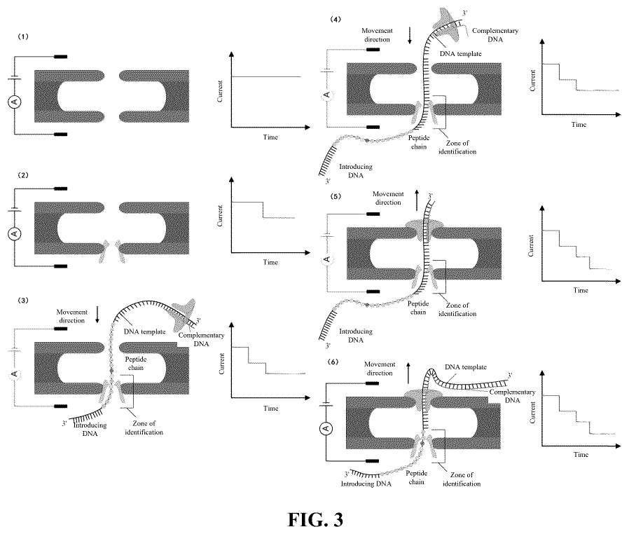

The combined nanopore protein sequencing method includes the following steps:
(1) Pretreatment of Si₃N₄/SiO₂/Si₃N₄ sandwich film chip: A Si₃N₄/SiO₂/Si₃N₄sandwich film chip is selected, and the Si₃N₄/SiO₂/Si₃N₄ sandwich thin film needs to be pretreated before processing. The specific pretreatment steps include: heating the Si₃N ₄/SiO₂/Si₃N₄ sandwich film in a piranha solution at a 90° C. water bath for 30 minutes to remove organic contamination and other impurities on the surface of the Si₃N₄/SiO₂/Si₃N₄ sandwich film.
(2) The Si₃N₄/SiO₂/Si₃N₄ sandwich film chip is milled by related processing methods to prepare nanopores: The pretreated Si₃N₄/SiO₂/Si₃N₄ sandwich film chip is processed by focused ion beam to obtain a through-hole structure.
(3) Preparation of “hole-cavity-hole” structure by etching silicon oxide cavities with buffered oxide etching solution (BOE): The Si₃N₄/SiO₂/Si₃N₄ sandwich film chip with nanopores prepared in Step (2) is tilted in BOE solution and etched for 3-5 minutes to obtain a SiO₂ cavity with a certain diameter, and a Si₃N₄/SiO₂/Si₃N₄ sandwich film chip with “hole-cavity-hole” structure is obtained.
(4) The solid nanopores embedded in the “hole-cavity-hole” structure are driven by the electric field of the functional protein: The Si₃N₄/SiO₂/Si₃N₄ sandwich film chip of Step (3) is washed with a piranha solution and loaded into the liquid pool chamber. The electrolyte is filled on both sides of the liquid pool chamber, and a bias voltage is applied. As shown in FIG. 1, the chip material of the sandwich film in FIG. 1 is Si₃N₄/SiO₂/Si₃N₄, and the lower hole in the “hole-cavity-hole” structure is a protein MspA with high-resolution reading ability. The pore is a functional protein phi29DNAP that controls the translocation rate of biomolecules, the configured MspA solution is added to one end of the liquid cell, and MspA is embedded into one of the two solid nanopores driven by the electrophoresis force. Then, the peptide-modified DNA molecule and the complementary DNA of the molecular template chain of the peptide-modified DNA are mixed and annealed, so that the tail end of the peptide-modified DNA molecular template chain is partially double-stranded. Then, the protein phi29DNAP with the function of a biomolecular motor is added to bind to the double-stranded part at the end of the template chain. Subsequently, it is added to the other end of the liquid pool. Driven by the electrophoresis force, the peptide-modified DNA molecule and the motor-functioning protein were combined into and embedded in another solid nanopore.
(5) Protein molecular read length detection (FIG. 2): The DNA peptide modification part is controlled by the process of synthesizing the single-stranded part of the DNA template chain. By using the protein MspA with high-resolution reading ability, the sequence of the target egg peptide chain is read and detected. When the peptide sequence enters the reading protein MspA, the reading of the peptide sequence shows a change in the current signal (FIG. 3). Then, the peptide modified on the DNA chain enters the cavity and moves to the phi29DNAP, the cavity distance between the two proteins determines the reading length of the peptide. When the distance of the cavity is sufficient, the method can complete the long-distance, single-molecule reading of a single protein molecule and realize the sequencing of the complete protein chain.

FIG. 3 is the current change diagram of each stage of protein sequencing. When the peptide segment modified on the DNA chain enters the cavity and moves towards the driving protein, there is a layer of cavity between the two functional proteins, and the distance of the cavity can be determined according to the actual situation. It can effectively overcome the problem of reading length caused by the limitation of the nano-space of protein molecules, complete the long-distance, single-molecule reading of a single protein molecule, and realize protein sequencing.

Therefore, the present disclosure adopts the above-mentioned protein detection method based on a combined nanopore structure, which can effectively overcome the problem of protein detection reading length caused by the limitation of nanopore space. Meanwhile, compared with the method of embedding the combined protein of related functions into the phospholipid bilayer, embedding the functional protein into the solid nanopore, combining the advantages of the biological nanopore and the solid nanopore, not only reduces the strict requirements of the sensor on the detection environment, but also improves the resolution and stability.

Finally, it should be explained that the above embodiments are only used to explain the technical scheme of the present disclosure rather than restrict it. Although the present disclosure is described in detail with reference to the better embodiment, the ordinary technical personnel in this field should understand that they can still modify or replace the technical scheme of the present disclosure, and these modifications or equivalent substitutions cannot make the modified technical scheme out of the spirit and scope of the technical scheme of the present disclosure.

What is claimed is:

1. A protein detection method based on a combined nanopore structure, comprising the following steps:
1) construction of a protein detection device with a combined nanopore structure, comprising:
Step 1, processing a surface of a sandwich film chip and milling to form nano-through holes;
Step 2, preparation of a “hole-cavity-hole” structure, by tilting the sandwich film chip with nanopores prepared in Step 1 in BOE solution, and using a buffer oxide etchant to etch an intermediate layer to obtain a sandwich film chip with a “hole-cavity-hole” structure;
Step 3, embedding a functional protein in the sandwich film chip with the “hole-cavity-hole” structure using an electric field, by washing the sandwich film chip obtained in Step 2 with piranha solution and loading into a liquid pool chamber, filling two sides of the liquid pool chamber with the electrolyte containing two functional proteins, and applying a bias voltage, wherein, under action of electric field force, the two functional proteins are embedded in the solid nanopores on both sides of the sandwich film chip, thus obtaining the protein detection device based on the combined nanopore structure;
where the two functional proteins are functional proteins that control the translocation speed of biomolecules and proteins with high-resolution reading ability, respectively;
2) protein detection, comprising:
driving peptide-modified DNA molecules to read and detect peptide sequences in peptide-modified DNA molecules using functional proteins that control the translocation speed of biomolecules through proteins with high-resolution reading ability, wherein, when the peptide sequence enters the protein with high-resolution reading ability, a reading of the peptide sequence causes a change in a current signal.

2. The protein detection method based on the combined nanopore structure according to claim 1, wherein, when etching in Step 2, the volume of the middle layer cavity is adjusted by controlling the etching time, the etching time is 0.5 s-30 min, and the volume of the middle layer cavity is 100 nm³-1×10⁵ nm³.

3. The protein detection method based on the combined nanopore structure according to claim 1, wherein the functional proteins that control the rate of translocation of biomolecules comprise phi29DNAP, hel308, and ClpX helicase, and proteins with high-resolution reading ability comprise MspA, α-HL, SP1, aerolysin, Phi29, CsgG, SPP1, and FraC.
